(19) United States
(12) Patent Application Publication
(10) Pub. No.: US 2024/0325181 A1
(43) Pub. Date: Oct. 3, 2024

(54) STENT DELIVERY DEVICE

(71) Applicant: S&G BIOTECH, INC., Yongin-si (KR)

(72) Inventors: Sung Kwon KANG, Yongin-si (KR); Hyun CHOI, Yongin-si (KR); Ju Won KO, Yongin-si (KR); Seo Hee KANG, Hwaseong-si (KR)

(21) Appl. No.: 18/215,478

(22) Filed: Jun. 28, 2023

(30) Foreign Application Priority Data
Mar. 30, 2023 (KR) 10-2023-0041810

(51) Int. Cl. A61F 2/966

(52) U.S. Cl. CPC A61F 2/966; A61F 2250/0007

(57) ABSTRACT

The present disclosure relates to a stent delivery device comprising a protective case configured to have a certain length and have an inner space through which a guide wire passes, a mover configured to have an inner surface facing an outer surface of the protective case, be movable along the longitudinal direction of the protective case, and be penetrated at both ends, and a sheath connector configured to be connected to the mover to be linked to the mover, thereby being movable in the inner space of the protective case, through which the guide wire passes, and allow a control wire for contracting and expanding the stent connected to the guide wire to enter and exit.

### CROSS-REFERENCE TO RELATED APPLICATION(S)

This application claims priority to Korean Patent Application No. 10-2023-0041810 filed in the Korean Intellectual Property Office on Mar. 30, 2023, the disclosure of which is incorporated by reference herein in its entirety.

### TECHNICAL FIELD

The present disclosure relates to a stent delivery device, and more particularly, to a stent delivery device capable of adjusting the length to suit various body types of a recipient, minimizing side effects of a procedure, and enabling an accurate procedure.

### DISCUSSION OF RELATED ART

Bile is produced in the liver and stored and concentrated in the gallbladder, and the bile duct serves as a passage through which the bile flows into the duodenum.

Bile duct stenosis refers to a disease in which the bile duct is narrowed or blocked by pancreatitis, pancreatic cancer, cholangitis, cholangiocarcinoma, or a tumor thereof.

This narrowing of the bile duct can be treated with surgery, but recently, endoscopic stent insertion has been used.

In particular, stent insertion is widely performed when surgery is impossible due to malignant bile duct stenosis in terminal cancer patients.

However, the conventional stents for bile ducts had a problem in that food passing through the duodenum is frequently refluxed into the bile duct, thereby obstructing the flow of bile, which causes side effects due to cholangitis or malfunction of the stent.

Further, the conventional stent for bile ducts had a problem in that it was difficult to adjust the inserted length corresponding to the length of the bile duct, which was different for each to-be-treated person.

### SUMMARY

The present disclosure was derived to address the above issues, to provide a stent delivery device capable of adjusting the length to suit various body types of a recipient, minimizing side effects of a procedure, and enabling an accurate procedure.

Further, the present disclosure is to provide a stent delivery device that can obtain accurate and neat surgical results inside the bile duct.

In order to achieve the above object, the present disclosure may provide a stent delivery device comprising a protective case configured to have a certain length and have an inner space through which a guide wire passes, a mover configured to have an inner surface facing an outer surface of the protective case, be movable along the longitudinal direction of the protective case, and be penetrated at both ends, and a sheath connector configured to be connected to the mover to be linked to the mover, thereby being movable in the inner space of the protective case, through which the guide wire passes, and allow a control wire for contracting and expanding the stent connected to the guide wire to enter and exit.

Further, the stent delivery device may further comprise a first cap configured to be detachably coupled to a trailing end of the protective case, allow the guide wire and the control wire to pass through and enter and exit, and at the same time, prevent the mover from being separated from the trailing end of the protective case; and a second cap configured to be detachably coupled to a leading end of the protective case.

Further, the protective case may be adjustable in length.

Further, the protective case may further comprise a housing configured to be penetrated through both ends, an inspection slot configured to be cut into the outer surface of the housing along the longitudinal direction of the housing, a first coupling step configured to be formed stepwise at the trailing end of the housing, and a second coupling step configured to be formed stepwise at the leading end of the housing, in which the guide wire can enter and exit through the first coupling step and the housing.

Further, the stent delivery device may further comprise a first cap configured to be detachably coupled to one end of the protective case, allow the guide wire and the control wire to pass through and enter and exit, and at the same time prevent the mover from being separated from one end of the protective case, and a stopper configured to contact a front end of the first cap, contacting or spaced apart from the front end of the mover with respect to the trailing end of the fixed mover, and be detachable from the outer surface of the protective case.

Further, the stent delivery device may further comprise a first engaging flange protruding along the edge of the front end of the first cap to prevent the trailing end of the mover from being separated from the protective case, an inspection slot configured to be cut into an outer surface of the protective case along the longitudinal direction of the protective case, and recessed grooves configured to be formed on the outer surface of the protective case to be disposed on the outer edges of both sides of the inspection slot, and in which the stopper is coupled and seated.

Further, the stopper may comprise a main body configured to be in contact with a surface of the front end of the mover, an engaging cutout configured to be formed by incision from one edge of the main body toward the other edge of the main body and have an inner edge facing the outer surface of the protective case, and a handle configured to protrude from the other edge of the main body and be provided to be graspable.

Further, each of the opposite sides of the inner edge of the engaging cutout may be recessed on the outer surface of the protective case and is coupled and seated in engaging grooves disposed on the outer edges of both sides of the inspection slot.

Further, the stopper may further comprise a reinforcing rib configured to protrude along the edge of the main body and a hook configured to protrude from both sides of the entrance of the inner edge of the engaging cutout to face each other, be recessed on the outer surface of the protective case and be engaged and fixed in hooking grooves disposed on the outer edges of both sides of the inspection slot.

Further, the mover may comprise a first divided part configured to occupy one-half of an inner surface facing the outer surface of the protective case, a second divided part configured to occupy the other half of the inner surface facing the outer surface of the protective case so as to have an inner surface facing the outer surface of the protective case by being coupled with the first divided part, and a tip protruding slope portion configured to be formed along the edge of the leading end of each of the first divided part and the second divided part so that separation from the protective case is prevented by a second cap detachably coupled to the leading end of the protective case, in which the sheath connector may be movable along the protective case together with the first divided part and the second divided part while being in communication with each other through a coupling part of the first divided part and the second divided part.

Further, the mover may further comprise a tube through-hole which is cut at the coupling part of the first divided part and the second divided part to communicate with a flushing port of the sheath connector.

Further, the sheath connector may comprise a main tube configured to be movable along the inner space of the protective case and have both ends through which the guide wire passes for entry and exit, and a flushing port configured to protrude inclinedly from an outer circumferential surface of the main tube, be exposed through a tube through-hole by passing through the outer surface of the mover, and communicate with the inner space of the main tube to enable entry and exit of the control wire, in which the flushing port may be exposed through an inspection slot cut into the outer surface of the protective case along the length direction of the protective case.

Further, the sheath connector may further comprise an O-ring configured to be embedded in the main tube and through which the guide wire passes, and an O-ring cover configured to be embedded in the main tube, through which the guide wire passes, and disposed between the O-ring and the leading end of the main tube.

Further, the housing may further comprise a plurality of units configured to detachably coupled between the trailing end of the housing where the first coupling step is formed and the leading end of the housing where the second coupling step is formed and be arranged in a line between the first coupling step and the second coupling step, in which both ends of each of the plurality of units are detachable from each other, in which the overall length of the protective case may be adjustable by adding or subtracting the number of the plurality of units.

Further, the stent delivery device may further comprise a second engaging flange configured to protrude along the edge of the trailing end of the second cap to prevent the leading end of the mover from escaping from the protective case.

According to the present disclosure having the configuration as described above, the length can be adjusted according to various body types of the to-be-treated person, and the side effects of the procedure can be minimized, and the precise procedure can be performed.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 illustrates a perspective conceptual view showing the overall appearance of a stent delivery device according to an embodiment of the present disclosure;

FIG. 2 illustrates an exploded perspective conceptual view showing the overall coupling relationship of the stent delivery device according to an embodiment of the present disclosure;

FIG. 3 illustrates a perspective conceptual view showing a length-adjustable state of a stent delivery device according to various embodiments of the present disclosure;

FIG. 4 illustrates a perspective conceptual view showing the operating state of the stent delivery device according to an embodiment of the present disclosure;

FIGS. 5 and 6 illustrate conceptual views showing the internal structure and the coupling structure of the main parts of the stent delivery device according to an embodiment of the present disclosure; and

FIG. 7 illustrates a conceptual side view sequentially illustrating the operating process of a stent delivery device according to an embodiment of the present disclosure.

### DETAILED DESCRIPTION OF EXEMPLARY EMBODIMENTS

The terms used in this specification will be briefly described, and the present disclosure will be described in detail. The advantages and characteristics of the present disclosure, and methods for achieving them become clear with reference to the embodiments described later in detail in conjunction with the drawings.

However, the present disclosure is not limited to the embodiments disclosed below and is implemented in various diverse forms.

In this specification, this embodiment is provided to complete the present disclosure, and to completely inform those skilled in the art of the scope of the present disclosure to which the present disclosure belongs.

Further, the present disclosure is only defined by the scope of the claims.

Thus, in some embodiments, well-known components, well-known operations, and well-known techniques have not been described in detail in order to avoid obscuring the interpretation of the present disclosure.

Further, like reference numerals designate like elements throughout the specification, and terms used (referred to) in this specification are for describing embodiments and are not intended to limit the present disclosure.

In this specification, the singular also includes the plural unless specifically stated in the phrase, and components and operations referred to as ‘comprising (or including)’ do not exclude the presence or addition of one or more other components and operations.

Unless otherwise defined, all terms (including technical and scientific terms) used in this specification may be used in a meaning commonly understood by those of ordinary skill in the art to which the present disclosure belongs.

In addition, terms defined in commonly used dictionaries are not interpreted ideally or excessively unless they are defined.

Hereinafter, preferred embodiments of the present disclosure are described with reference to the accompanying drawings.

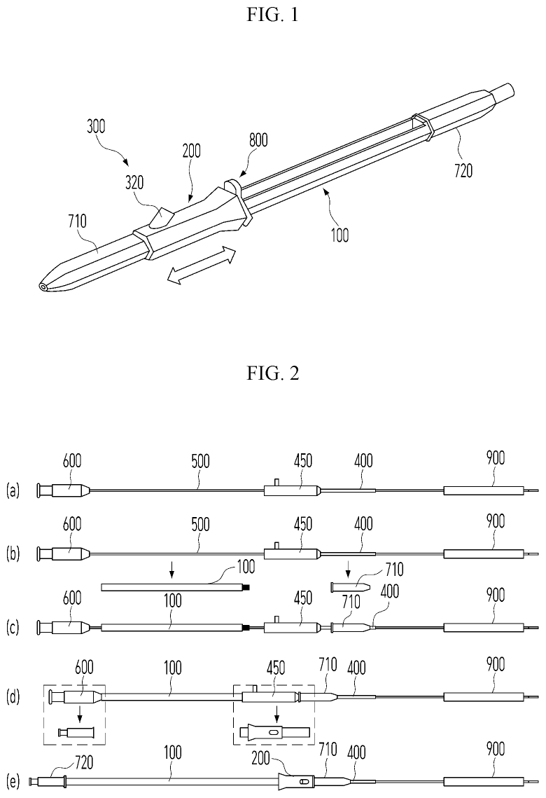

First of all, FIG. 1 illustrates a perspective conceptual view showing the overall appearance of a stent delivery device according to an embodiment of the present disclosure.

Further, FIG. 2 illustrates an exploded perspective conceptual view showing the overall coupling relationship of the stent delivery device according to an embodiment of the present disclosure.

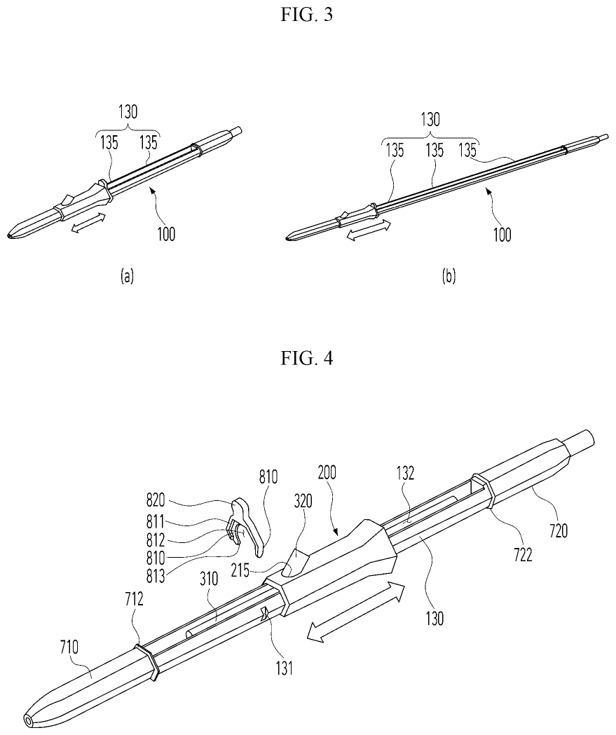

Further, FIG. 3 illustrates a perspective conceptual view showing a length-adjustable state of a stent delivery device according to various embodiments of the present disclosure.

Further, FIG. 4 illustrates a perspective conceptual view showing the operating state of the stent delivery device according to an embodiment of the present disclosure.

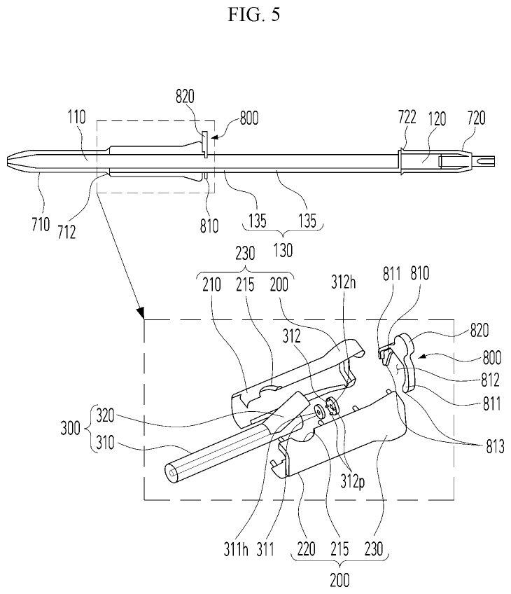

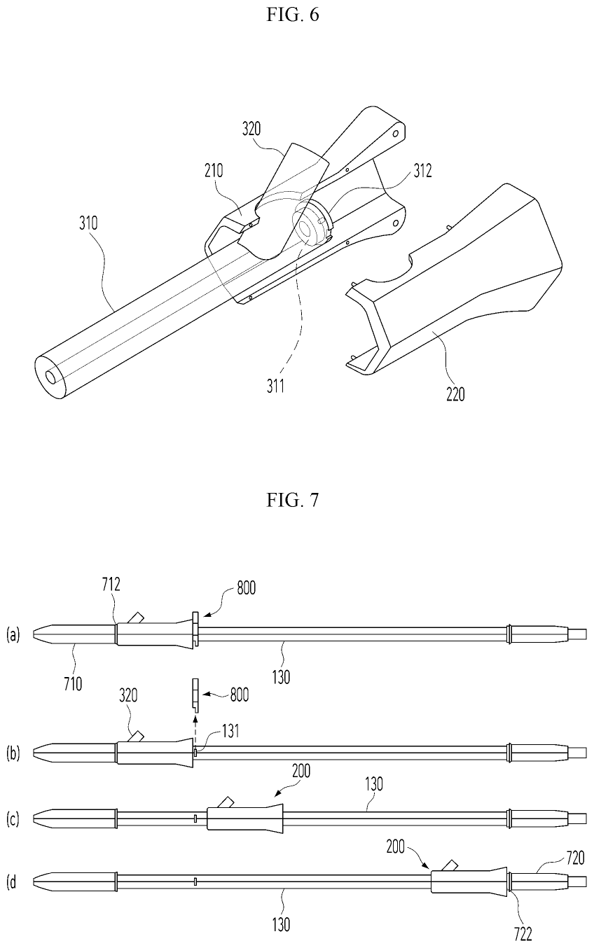

Further, FIGS. 5 and 6 illustrate conceptual views showing the internal structure and the coupling structure of the main parts of the stent delivery device according to an embodiment of the present disclosure.

Further, FIG. 7 illustrates a conceptual side view sequentially illustrating the operating process of a stent delivery device according to an embodiment of the present disclosure.

As shown in FIGS. 1 and 2, an embodiment including the protective case 100, the mover 200, and the sheath connector 300 may be applied to the present disclosure.

First, the protective case 100 has a certain length and has an inner space through which the guide wire 500 passes.

The mover 200 is a member that has an inner surface facing an outer surface of the protective case 100, is movable along the longitudinal direction of the protective case 100, and passes at both ends.

The sheath connector 300 is connected to the mover 200 and is linked to the mover 200 to be movable in the inner space of the protective case 100, through which the guide wire 500 passes, and allow a control wire (not shown) for contracting and expanding the stent 900 (See FIG. 2) connected to the guide wire 500 to enter and exit.

The above embodiments can be applied to the present disclosure, and the following various embodiments can also be applied thereto.

First, the guide wire 500 is guided by a sheath tube 400 made of polyimide as shown in FIG. 2, a sheath handle 450 is disposed on the sheath tube 400 to guide the entry and exit of the guide wire 500 together with the sheath tube 400, and a pusher handle 600 is provided at the leading end of the guide wire 500.

Here, the guide wire 500 is formed in a pipe shape made of steel or a steel alloy material and is provided to guide the movement of the sheath handle 450.

Meanwhile, the protective case 100 may perform the following roles.

First, the protective case 100 serves to guide the sheath connector 300 to move in a straight line.

That is, in the conventional stent delivery device, since the pusher handle 600 and the sheath handle 450 were connected only with the guide wire 500 as shown in FIGS. 2a and 2b, so there was a problem of being very vulnerable to bending during the procedure.

Therefore, the protective case 100 according to the present disclosure compensates for the weakness of the bending described above as shown in FIGS. 2a and 2b, and it serves to guide the sheath connector 300 to move in a straight line.

Further, the protective case 100 is prepared to unify the direction of stent deployment.

That is, in the conventional stent delivery device, for placement of the stent connected to the sheath tube 400 passing through the guide wire 500 connecting the pusher handle 600 and the sheath handle 450, the stent could be placed in the target lesion only by pushing the pusher handle 600 toward the sheath handle 450.

In contrast, in the protective case 100 according to the present disclosure, the stent can be placed on the target lesion only by pulling the mover to the second cap 720, which will be described later, in a state in which the protective case 100 is combined with the second cap 720 and integrally formed as shown in FIGS. 2d and 2e, thereby preventing an operator's error and inducing accurate stent placement.

Therefore, components including the protective case 100 according to the present disclosure, the first and second caps 710 and 720 and the mover 200, which will be described later are not inserted into the target lesion, that is, the body of the to-be-treated person, instead only the sheath tube 400 and the stent connected to this sheath tube 400 are inserted thereinto.

Meanwhile, the first cap 710 is detachably coupled to the trailing end of the protective case 100.

The first cap 710 is provided to allow the guide wire 500 and the control wire to pass through and enter and exit, and to play a role of preventing the mover 200 from escaping from one end of the protective case 100.

Meanwhile, the second cap 720 is detachably coupled to the leading end of the protective case 100.

The first cap 720 is provided to allow the guide wire 710 and the control wire to pass through and enter and exit, and to play a role in preventing the mover 200 from escaping from the leading end of the protective case 100.

Meanwhile, the protective case 100 can be adjusted in length as shown in FIG. 3 to fit the body shape of various to-be-treated person.

The length of the protective case 100 may be adjusted by adding or subtracting the number of units 135 constituting the housing 130 to be described later.

The protective case 100 may comprise the housing 130 whose both ends are penetrated and the inspection slot 132 cut into the outer surface of the housing 130 along the longitudinal direction of the housing 130.

The protective case 100 may further comprise the first coupling step 110 formed stepwise at the trailing end of the housing 130 and the second coupling step 120 formed stepwise at the leading end of the housing 130.

Here, the guide wire 500 may enter and exit through the first coupling step 110 and the housing 130.

In this case, the housing 130 may further comprises a plurality of units 135 which is detachably coupled between the trailing end of the housing 130 where the first coupling step 110 is formed and the leading end of the housing 130 where the second coupling step 120 is formed and are arranged in a line between the first coupling step 110 and the second coupling step 120.

Both ends of each of the plurality of units may be detached from each other, and the overall length of the protective case may be adjusted by adding or subtracting the number of the plurality of units.

Meanwhile, it may further comprise the second engaging flange 722 protruding along the edge of the trailing end of the second cap 720 to prevent the leading end of the mover 200 from escaping from the protective case 100.

Meanwhile, the stent delivery device may further comprise the stopper 800 contacting the leading end of the first cap 710, thereby contacting or spaced apart from the front end of the mover with respect to the trailing end of the fixed mover 200 and is detachable from the outer surface of the protective case 100.

Here, in order to implement the exact function of the stopper 800, it may further comprise the first engaging flange 712 protruding along the edge of the leading end of the first cap 710 to prevent the trailing end of the mover 200 from escaping from the protective case 100.

In this case, the inspection slot 132 may be cut into an outer surface of the protective case 100 along the longitudinal direction of the protective case 100.

Further, each of engaging grooves 131 (See FIG. 4) are recessed on the outer surface of the protective case 100 to be disposed of on the outer sides of both sides of the inspection slot 132, thereby providing a space and area in which the stopper 800 is coupled and seated.

This stopper 800 may comprise, as shown in FIGS. 4 and 5, the main body 810 in contact with a surface of the leading end of the mover 200, and the engaging cutout 812 formed by incision from one edge of the main body 810 toward the other edge of the main body 810 and having an inner edge facing the outer surface of the protective case 100.

The stopper 800 may comprise the handle 820 protruding from the other edge of the main body 810 and be provided to be graspable.

Here, each of the opposite sides of the inner edge of the engaging cutout 812 is recessed on the outer surface of the protective case 100 and is coupled and seated in engaging grooves 131 disposed on the outer edges of both sides of the inspection slot 132.

In this case, the stopper 800 may further comprise the reinforcing rib 811 protruding along the edge of the main body 811.

Further, the stopper 800 may further comprise a hook protruding from both sides of the entrance of the inner edge of the engaging cutout 812 to face each other, recessed on the outer surface of the protective case 100 to be engaged and fixed in engaging grooves 131 disposed on the outer edges of both sides of the inspection slot 132.

Meanwhile, referring more specifically with reference to the lower enlarged portion of FIG. 5, the mover 200 may comprise the first divided part 210 occupying one-half of an inner surface facing the outer surface of the protective case 100, and the second divided part 220 occupying the other half of the inner surface facing the outer surface of the protective case 100.

The second divided part 220 is coupled with the first divided part 210 so as to have an inner surface facing the outer surface of the protective case 100.

The mover 200 may comprise the tip protruding slope portion 230 formed along the edge of the leading end of each of the first divided part 210 and the second divided part 220 so that separation from the protective case 100 is prevented by the second cap 720 detachably coupled to the leading end of the protective case 100.

Therefore, the sheath connector 300 is movable along the protective case 100 together with the first divided part 210 and the second divided part 220 while being in communication with each other through a coupling part of the first divided part 210 and the second divided part 220.

Further, the mover 200 may further comprise a tube through-hole 215 which is cut at the coupling part of the first divided part 210 and the second divided part 220 to communicate with the flushing port 320 of the sheath connector 300.

That is, the tube through-hole 215 is formed by combining a cutout formed in an arc shape cut at the coupling part of the first divided part 210 and a cutout formed in an arc shape cut at the coupling part of the second divided part 220, thereby allowing communication of the flushing port 320.

Meanwhile, the sheath connector 300 may comprise the main tube 310 movable along the inner space of the protective case 100 and having both ends through which the guide wire 500 passes for entry and exit, and

the flushing port 320 protruding inclinedly from an outer circumferential surface of the main tube, exposed through a tube through-hole 215 by passing through the outer surface of the mover 200 and communicating with the inner space of the main tube 310 to enable entry and exit of the control wire.

Here, the flushing port 320 is exposed through the inspection slot 132 cut into the outer surface of the protective case 100 along the length direction of the protective case 100.

In this case, the sheath connector 300 may further comprise the O-ring 311 embedded in the main tube 310 and through which the guide wire 500 passes and the O-ring cover 312 embedded in the main tube 310, through which the guide wire 500 passes, and disposed between the O-ring 312 and the leading end of the main tube 310.

The O-ring 311 is preferably made of a silicone material that is harmless to the human body.

Flushing is performed by injecting saline solution through the flushing port 320 before the stent procedure, thereby filling the saline solution up to the front of the stent.

Therefore, the O-ring 311 serves as a check valve so as to prevent the saline solution injected during the flushing process from flowing backward, that is, to prevent the outflow of bodily fluid that may flow backward through the sheath tube 400 inside the body of the to-be-treated person.

Here, the O-ring hole 311h may be formed at the center of the O-ring 311 to allow the sheath tube 400 to pass therethrough.

In addition, the O-ring cover 312 is made of the same material as the sheath connector 300 and may be made of, for example, a polycarbonate material that has high durability and structural strength and is harmless to the human body.

The O-ring cover 312 serves to hold the O-ring 311 so that the O-ring 311 can maintain the initially installed position during the forward or backward movement of the mover 200, and to this end, it may be adhesively fixed to the main tube 310 as shown in FIG. 6.

Here, the cover hole 312h may be formed at the center of the O-ring cover 312 to allow the sheath tube 400 to pass therethrough.

In this case, the O-ring cover 312 may further comprise a plurality of fixing protrusions 312p protruding from one surface of the O-ring cover 312 and radially disposed around the cover hole 312h.

Therefore, the plurality of fixing protrusions 312p may be adhesively fixed to the main tube 310 as shown in FIG. 6.

Hereinafter, with reference to FIG. 7, the operation process of the stent delivery device according to an embodiment of the present disclosure is sequentially and briefly described.

The movement of the mover 200 to which the flushing port 320 is coupled is restricted by the stopper 800 coupled to the protective case 100 as shown in FIG. 7a.

The operator can separate the stopper 800 from the engaging grooves 131 of the protective case 100 as shown in FIG. 7b and move the mover 200 along the housing 130 of the protective case 100 as shown in FIG. 7c to the leading end of the housing 130 as shown in FIG. 7d.

That is, the operator inserts the endoscope (not shown) near the target lesion of the to-be-treated person, fixes it to the target lesion, and inserts the guide wire 500 into the endoscope to the target lesion.

Thereafter, the operator passes the guide wire 500 through the sheath tube 400 from the first cap 710 to the second cap 720 to push the stent connected to the sheath tube 400 into the body of the to-be-treated person.

Here, as described above, the only parts that enter the body of the to-be-treated person are the stent and the sheath tube 400.

Subsequently, when the stent reaches the target lesion, the operator operates the mover 200 to deploy the stent, that is, retreats the mover 200 toward the second cap 720 as shown in FIG. 7d.

Next, after the stent is completely deployed in the target lesion, the operator returns the mover 200, which has retreated to the second cap 720, to the first cap 710 as shown in FIG. 7a.

Finally, the operation is finished by pulling the stent delivery device including the guide wire 500 inside the endoscope, the protective case 100 to which the first and second caps 710 and 720 according to the present disclosure are coupled, and the mover 200, taking it out from the body of the to-be-treated person, and performing a series of processes such as suturing by the operator.

As described above, it can be seen that the basic technical idea of the present disclosure is to provide a stent delivery device capable of adjusting the length according to various body types of a to-be-treated person, minimizing side effects of the procedure, and enabling accurate surgery.

Within the scope of the basic technical idea of the present disclosure, many other modifications and applications can be made by those skilled in the art.

What is claimed is:

1. A stent delivery device comprising:
a protective case configured to have a certain length and have an inner space through which a guide wire passes;
a mover configured to have an inner surface facing an outer surface of the protective case, be movable along the longitudinal direction of the protective case, and be penetrated at both ends; and
a sheath connector configured to be connected to the mover to be linked to the mover, thereby being movable in the inner space of the protective case, through which the guide wire passes, and allow a control wire for contracting and expanding the stent connected to the guide wire to enter and exit.

2. The stent delivery device of claim 1, further comprising:
a first cap configured to be detachably coupled to a trailing end of the protective case, allow the guide wire and the control wire to pass through and enter and exit, and at the same time prevent the mover from being separated from the trailing end of the protective case; and
a second cap configured to be detachably coupled to a leading end of the protective case.

3. The stent delivery device of claim 1, wherein the protective case is adjustable in length.

4. The stent delivery device of claim 1, wherein the protective case further comprises:
a housing configured to be penetrated through both ends;
an inspection slot configured to be cut into the outer surface of the housing along the longitudinal direction of the housing;
a first coupling step configured to be formed stepwise at the trailing end of the housing; and
a second coupling step configured to be formed stepwise at the leading end of the housing,
wherein the guide wire can enter and exit through the first coupling step and the housing.

5. The stent delivery device of claim 1, further comprising:
a first cap configured to be detachably coupled to one end of the protective case, allow the guide wire and the control wire to pass through and enter and exit, and at the same time prevent the mover from being separated from one end of the protective case; and
a stopper configured to contact a front end of the first cap, contacting or spaced apart from the front end of the mover with respect to the trailing end of the fixed mover, and be detachable from the outer surface of the protective case.

6. The stent delivery device of claim 5, further comprising:
a first engaging flange protruding along the edge of the front end of the first cap to prevent the trailing end of the mover from being separated from the protective case;
an inspection slot configured to be cut into an outer surface of the protective case along the longitudinal direction of the protective case; and
recessed grooves configured to be formed on the outer surface of the protective case to be disposed on the outer edges of both sides of the inspection slot, and in which the stopper is coupled and seated.

7. The stent delivery device of claim 5, wherein the stopper comprises a main body configured to be in contact with a surface of a front end of the mover;
an engaging cutout configured to be formed by incision from one edge of the main body toward the other edge of the main body and have an inner edge facing the outer surface of the protective case; and
a handle configured to protrude from the other edge of the main body and be provided to be graspable.

8. The stent delivery device of claim 7, wherein each of opposite sides of the inner edge of the engaging cutout is recessed on the outer surface of the protective case and is coupled and seated in engaging grooves disposed on the outer edges of both sides of the inspection slot.

9. The stent delivery device of claim 7, wherein the stopper further comprises:
a reinforcing rib configured to protrude along the edge of the main body; and
a hook configured to protrude from both sides of the entrance of the inner edge of the engaging cutout to face each other, be recessed on the outer surface of the protective case and be engaged and fixed in engaging grooves disposed on the outer edges of both sides of the inspection slot.

10. The stent delivery device of claim 1, wherein the mover comprises a first divided part configured to occupy one-half of an inner surface facing the outer surface of the protective case;
a second divided part configured to occupy the other half of the inner surface facing the outer surface of the protective case so as to have an inner surface facing the outer surface of the protective case by being coupled with the first divided part; and
a tip protruding slope portion configured to be formed along the edge of the leading end of each of the first divided part and the second divided part so that separation from the protective case is prevented by a second cap detachably coupled to the leading end of the protective case,
wherein the sheath connector is movable along the protective case together with the first divided part and the second divided part while being in communication with each other through a coupling part of the first divided part and the second divided part.

11. The stent delivery device of claim 10, wherein the mover further comprises a tube through-hole which is cut at the coupling part of the first divided part and the second divided part to communicate with a flushing port of the sheath connector.

12. The stent delivery device of claim 1, wherein the sheath connector comprises:
a main tube configured to be movable along the inner space of the protective case and have both ends through which the guide wire passes for entry and exit; and
a flushing port configured to protrude inclinedly from an outer circumferential surface of the main tube, be exposed through a tube through-hole by passing through the outer surface of the mover and communicate with the inner space of the main tube to enable entry and exit of the control wire, wherein the flushing port is exposed through an inspection slot cut into the outer surface of the protective case along the length direction of the protective case.

13. The stent delivery device of claim 12, wherein the sheath connector further comprises:
an O-ring configured to be embedded in the main tube and through which the guide wire passes; and
an O-ring cover configured to be embedded in the main tube, through which the guide wire passes, and disposed between the O-ring and the leading end of the main tube.

14. The stent delivery device of claim 4, wherein the housing further comprises a plurality of units configured to detachably coupled between the trailing end of the housing where the first coupling step is formed and the leading end of the housing where the second coupling step is formed and be arranged in a line between the first coupling step and the second coupling step,
wherein both ends of each of the plurality of units are detachable from each other, and
wherein the overall length of the protective case is adjustable by adding or subtracting the number of the plurality of units.

15. The stent delivery device of claim 2, further comprising a second engaging flange configured to protrude along the edge of the trailing end of the second cap to prevent the leading end of the mover from escaping from the protective case.
